(19) United States
(12) Patent Application Publication
(10) Pub. No.: US 2022/0354249 A1
(43) Pub. Date: Nov. 10, 2022

(54) MODULAR ADJUSTABLE STORAGE ORGANIZER

(71) Applicants: Gail Winifred Nichols, Tamarac, FL (US); Richard Sutton Cherry, Tamarac, FL (US)

(72) Inventors: Gail Winifred Nichols, Tamarac, FL (US); Richard Sutton Cherry, Tamarac, FL (US)

(21) Appl. No.: 17/737,059

(22) Filed: May 5, 2022

Related U.S. Application Data
(60) Provisional application No. 63/186,011, filed on May 7, 2021.

(51) Int. Cl. A47B 57/10; A47B 47/05; A47B 47/04

(52) U.S. Cl. CPC A47B 57/10; A47B 47/05; A47B 47/047

(57) ABSTRACT

An improved storage organizer (10) having a plurality of vertical modular side panels (30) providing support, adjustability of size, location and storage capacity for a plurality of horizontal, adjustable shelf rails (70), adjustable, extendable, pull-out shelves (20) and encoded labels (25A) enabling identification of stored items within upright freezers and refrigerators in the first embodiment. A user of the organizer (10) can assemble the organizer without tools in the first embodiment to suit various models, dimensions and inner configuration of the storage space and anticipated weight and sizes of items stored on the shelves. A user of the organizer can track quantity and content of stored items by means of encoded labels (25A) inserted in shelf label holders (25) and in or on packages containing stored items, both visually and by means of remote inventory control systems. Thus, a considerably more versatile, durable, modular and efficient storage organizer is provided—that can be adjusted to provide more or less storage capacity in most models of upright freezers and refrigerators in the first embodiment as well as in other cold, heated or room-temperature storage compartments in other embodiments with ease of tracking quantities and locations, viewing, adding, removing, rearranging, identifying and retrieving a variety of stored items.

### CROSS-REFERENCE TO RELATED APPLICATIONS

This application claims the benefit of provisional patent application Ser. No. 63/186,011, filed May 7, 2021 by the present inventors

### FEDERALLY SPONSORED RESEARCH

Nonapplicable.

### SEQUENCE LISTING

Nonapplicable.

### TECHNICAL FIELD

The present invention relates to storage organization, and more specifically to enclosed compartments.

### BACKGROUND:

Conventional storage systems in enclosed compartments, specifically in upright freezers and refrigerators, are inefficient and inconvenient as they limit the user to arranging and stacking items on a restricted number of manufacturer-installed fixed shelves, further limited by a variety of interior fixed protrusions such as ducts, lights and temperature controls. Said fixed shelves do not provide means to increase load-bearing capacity or to be easily repositioned within the compartment.

As well, users often have difficulty in locating stored items within stacked groups since conventional fixed shelving does not have means to affix labels or other identifiers of stored items. This leads to forgotten items that are stored past their expiry date and deteriorate beyond usability and are thus often wasted.

So, manufacturers of freezers and refrigerators and of storage organizers for freezers and refrigerators have long sought means of effectively adjusting the location of and access to stored food items for greater storage efficiency.

### DESCRIPTION OF PRIOR ART:

The following is a tabulation of some prior art that presently appears relevant:

U.S. Pat. No. 10,139,153 to Furr (2018Nov. 27)

U.S. Pat. No. 9,528,753 to Conner (2016Dec. 27)

U.S. Pat. No. 4,191,437 to Funke (1980Mar. 4)

U.S. Pat. No. 4,944,566 to Carper (1990Jul. -31

While the need for improved cost-effective storage efficiency has long been sought after, the prior art food storage systems have left many areas available for improvement. For example, many limit themselves to a portion of food storage in a refrigerator and do not include the full scope of storage organization for refrigerators and freezers.

Furr discloses a kit of stackable modular assemblies that includes one or more bins fashioned after drawers that open with a pull-out drawer from the front, for organizing and storing of food items within a refrigerator or cooling unit.

Conner discloses three embodiments including 1) rotatable shelves for refrigerators with stationary or fixed shelf including a rotatable disk mounted on a bearing retainer having bearings for manual 360-degree rotation as desired; and, 2) a quarter-round pivotable shelf attached to a stanchion to enable the shelf to be manually pivoted 90 degrees; and, 3) a rotatable disk in communication with a drive motor for rotation by a control switch.

Funke discloses a refrigerator storage system characterized by circular tracks located on shelving throughout the refrigerator for receiving rotatable shelves for selective storage of food products.

Carper discloses a refrigeration apparatus accessory storage system mounting to a refrigeration apparatus shelf.

In conclusion, insofar as we are aware, no storage organizer formerly developed for upright refrigerators and freezers provides adjustability of location and capacity and weight-bearing support as well as item identification to a user without the defects of fixed shelving, fixed rails, fixed frames and constrained structures.

### SUMMARY

In accordance with one embodiment, an improved modular storage organizer within an enclosed compartment, particularly in a cold environment of upright freezers and refrigerators for homes and commercial food service, comprises modular, adjustable rigid vertical and elongated horizontal supports having the structural strength and means to interconnect with adjustable, extendable, rigid pull-out shelves as replacement for conventional fixed shelves in upright freezers and refrigerators to allow users to adjust location, identification and weight- bearing capacity of stored items.

Thus, some embodiments provide means to enable a user to position the organizer inside an enclosed compartment as a free-standing unit as well as fastened to the wall, floor and/or ceiling. Some embodiments provide means to move the adjustable shelves among a plurality of horizontal supports to accommodate varying dimensions and weights of stored items as well as varying dimensions of depth, width and height inside various models of upright freezers and refrigerators and other enclosed environments while accommodating fixed inside protrusions of said models. Shelves also have means for connecting varying flexible and rigid, durable, washable, encoded labels in a plurality of locations to identify stored items visually and by means of electronic inventory control systems.

### Advantages

Accordingly, several advantages of one or more aspects are as follows.
a) to provide an improved storage organizer,
b) to provide means of increasing ease and convenience to the user of locating, identifying, supporting, positioning and retrieving stored items inside an enclosed compartment,
c) to provide means of accommodating fixed protrusions inside the cavity of a variety of makes and models of upright freezers and refrigerators,
d) to provide an organizer with means to increase storage efficiency and improve load- bearing capacity of upright freezers and refrigerators, and
e) to provide an organizer that improves quality of stored items while reducing associated waste.
Thus, some embodiments provide a storage organizer that is more efficient, durable and adaptable than prior-art organizers. These and other benefits of one or more aspects will become apparent from a consideration of the ensuing description.

### DRAWINGS—FIGURES-FIRST EMBODIMENT

In the drawings, closely related figures have the same number but different alphabetic suffixes.

FIG. 1 schematically illustrates an assembled organizer constructed in accordance with the first embodiment

FIG. 2 schematically illustrates an adjustable, extendable, pull-out shelf assembly shown in FIG.

FIG. 3 schematically illustrates an organizer left-side panel shown in FIG. 1

FIG. 4 schematically illustrates a post segment of the organizer side panel shown in FIG. 1 and FIG. 3

FIGS. 5, 5A, 5B, and 5C schematically show various aspects of a universal crossbar design positioned in different support applications: stabilizer, header, and footer shown in in FIG. 1 and FIG. 3

FIG. 6 schematically illustrates a discrete prong of the organizer side panel shown in FIG. 1 and FIG. 3

FIG. 7 schematically illustrates a shelf rail of the organizer shown in FIG. 1 and FIG. 3

REFERENCE NUMERALS OF THE DRAWINGS

| | | | | | |
|---|---|---|---|---|---|
| 10 | organizer | 25D | label holder | 51 | spacer bolt |
| | assembled | | hole | | head |
| 20 | shelf | 29 | post binder | 51A | spacer bolt |
| | assembled | 30 | side panel | 51B | hex nut for |
| 21 | base plate | | assembled | | stabilizer |
| 21A | base plate | 40 | post segment | 51C | hex nut for |
| | positioning | 41 | Integrated | | header |
| | hole | | prong | 52 | top security |
| 21B | base plate | 42 | top security | | slot |
| | positioning | | slot | 53A | top prong |
| | slot | 43 | top rail | | slot |
| 21C | base plate | | platform | 53B | bottom |
| | front | 44 | center security | | prong slot |
| | gripping | | slot | 55A | interlocking |
| | flange | 45 | wall | | surface |
| 21D | base plate | | mounting | 55B | wall brace |
| | hole- | | hole | | surface |
| | label holder | 45A | floor/ceiling | 55C | ceiling/floor |
| 22 | left side | | mounting | | brace surface |
| | extender | | hole | 56A | bolt hole |
| 22A | left side | 45B | prong | 56B | bolt hole |
| | extender | | interlocking | 57A | prong slot |
| | positioning | | hole | 57B | prong slot |
| | hole | 46 | rail gripping | 57C | prong slot- |
| 22B | left side | | grooves | | flanged |
| | extender | 47 | outward | 60 | discrete |
| | rail flange | | tapered | | prong |
| 23 | right side | | nesting flange | 64 | discrete |
| | extender | 48 | bottom open- | | prong |
| 23A | right side | | platform rail | | header |
| | extender | | gripping | | flange |
| | positioning | | grooves | 70 | shelf rail |
| | hole | 49 | post | 71 | extension |
| 23B | right side | | assembled | | security |
| | extender | 50 | crossbar- | | lock hole |
| | rail flange | | universal | | |
| 24 | back | | design | | |
| | extender | 50A | crossbar | | |
| 24A | back | | stabilizer | | |
| | extender | 50B | crossbar | | |
| | positioning | | header | | |
| | hole | 50C | crossbar | | |
| 24B | back | | footer | | |
| | extender | | | | |
| | flange | | | | |
| 24C | back | | | | |
| | extender | | | | |
| | security tab | | | | |
| 25 | label holder | | | | |
| 25B | label | | | | |

### DETAILED DESCRIPTION—FIRST EMBODIMENT

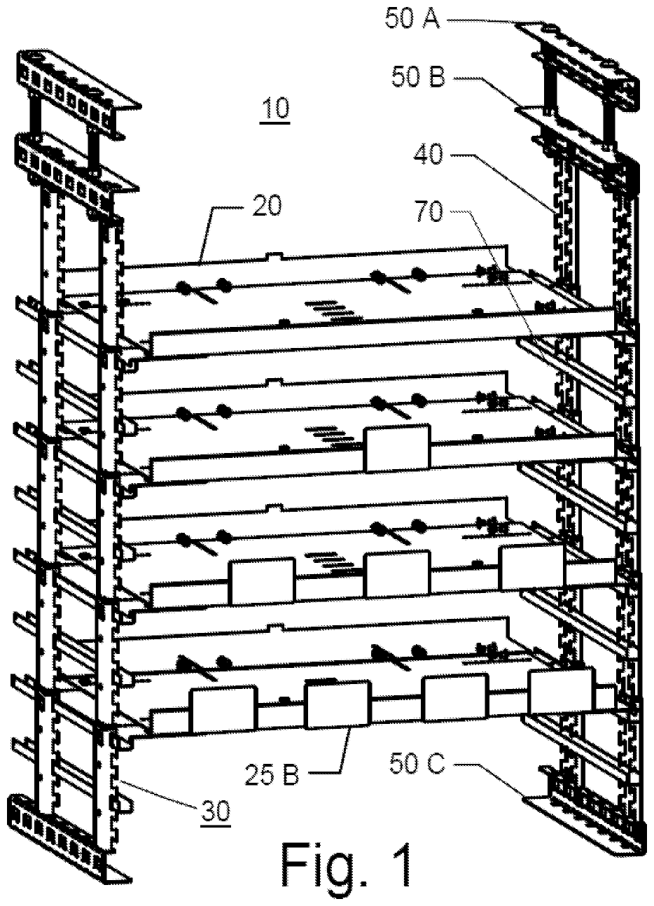

FIG. 1 schematically illustrates an overall view of the assembled organizer 10 of the first embodiment. The organizer comprises two parallel assembled vertical side panels 30, on the left-side and right-side of the organizer, each having modular, interlocking components including: post segments 40, horizontal stabilizer 50A, horizontal header 50B, horizontal footer 50C; horizontal elongated shelf rails 70, and adjustable, extendable rigid pull-out shelves 20 each optionally having one or more front-positioned labels 25B.

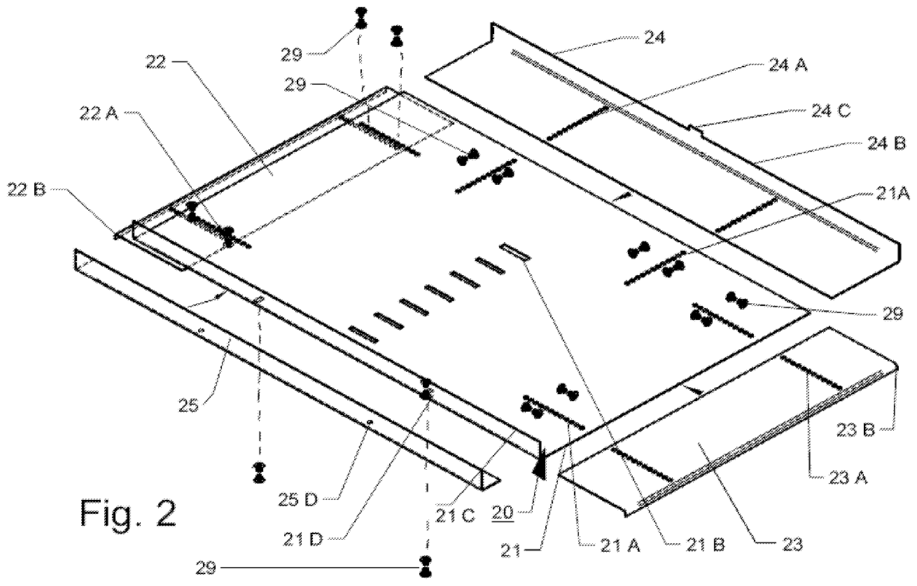

FIG. 2 schematically illustrates an overall view of the assembled modular extendable shelf 20 as shown in FIG. 1. Shelf 20 consists of sheets of rigid material that can be made of, but not limited to, thermoplastic polymer material, including synthetic resin, polypropylene, polyester, and ABS (acrylonitrile butadiene styrene).

Shelf 20 comprises the base plate 21, the left-side extender 22 shown underneath the left side of the base plate 21, the right-side extender 23 shown as unattached to the base plate 21 and the back extender 24 shown unattached to the base plate 21. The left-side extender 22 has a plurality of spaced positioning holes 22A and a rail flange 22B angled down. Similarly, the right- side extender 23 has a plurality of spaced positioning holes 23A and a rail flange 23B angled down. The base plate 21 has a plurality of spaced positioning holes 21A which connect with the positioning holes in the left-side extender 22 and right-side extender 23, by means of post binders 29 inserted in the aligned corresponding spaced positioning holes to thereby fit the width of the space between the left-side panel 30 and right-side panel 30.

The shelf base plate 21 connects with the back extender 24 by means of post binders 29 inserted in the base plate spaced positioning holes 21A aligned with the back extender spaced positioning holes 24A. The back extender 24 includes a back extender security flange 24B that is angled upwards, having a rigid security tab 24C with means to interlock with the base plate positioning slots 21B.

Each shelf 20 has two label holder holes 25D as means to attach the optional front label holder 25 that interlocks with the shelf base plate front gripping flange 21C by means of post binders 29 inserted in the label holder holes 25D. The label holder 25 has means for inserting a plurality of optional front-facing labels 25B (shown in FIG. 1) that are waterproof, freeze-proof, attractive, and easy-to-read to visually identify items on said shelf 20 by means of printed text and also electronically by encoding for remote inventory control.

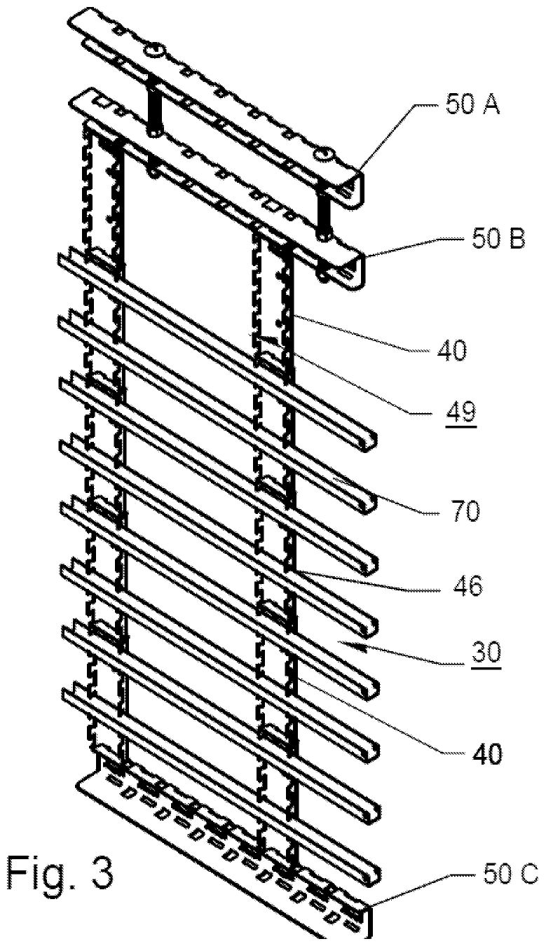

FIG. 3 schematically illustrates an overall view of the left side panel 30 consisting of rigid material that is structurally strong, durable and can be corrosion resistant, that can be made of, but not limited to, metal such as steel and aluminum, and wood, and thermoplastic polymer material. The side panel 30 comprises interlocking components including: a plurality of assembled posts 49 comprising a plurality of interlocking post segments 40; stabilizer 50A, header 50B, footer 50C; and shelf rails 70.

The shelf rail 70 interlocks with the post segment 40 by means of integrated parallel rail gripping grooves 46.

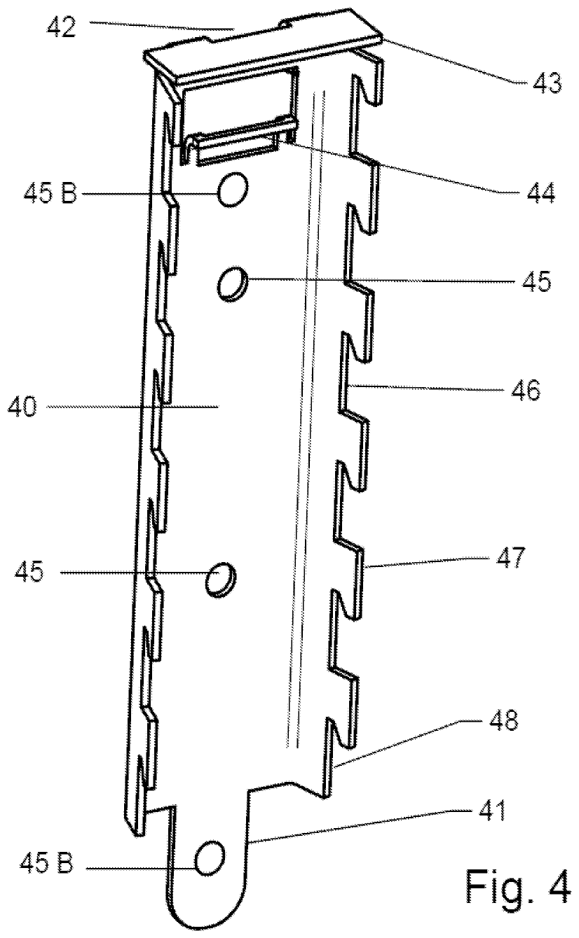

FIG. 4 is a perspective view of the post segment 40 shown in FIG. 1 and FIG. 3. Each upper post segment 40 interlocks with the lower post segment 40 by means of the integrated prong 41 on the bottom end of the upper post segment 40 that slides into the lower post segment 40 by means of the top security slot 42 and further extends to the center security slot 44. Each post segment 40 has a top rail platform 43 that augments the rail gripping grooves 46 and bottom open-platform rail gripping grooves 48, outward tapered nesting flanges 47 and the bottom integrated prong 41. The wall mounting holes 45 and the prong interlocking holes 45B are provided for optional use in this and other embodiments.

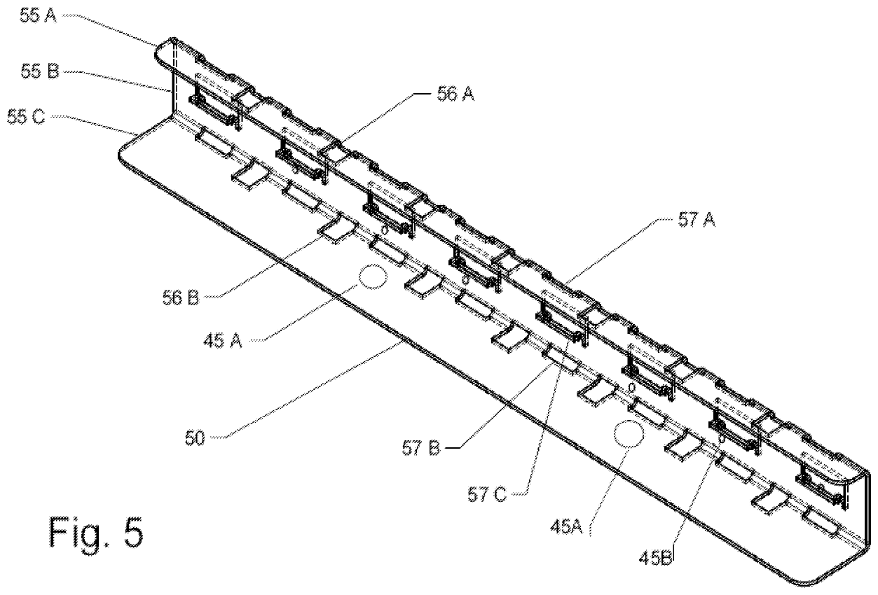

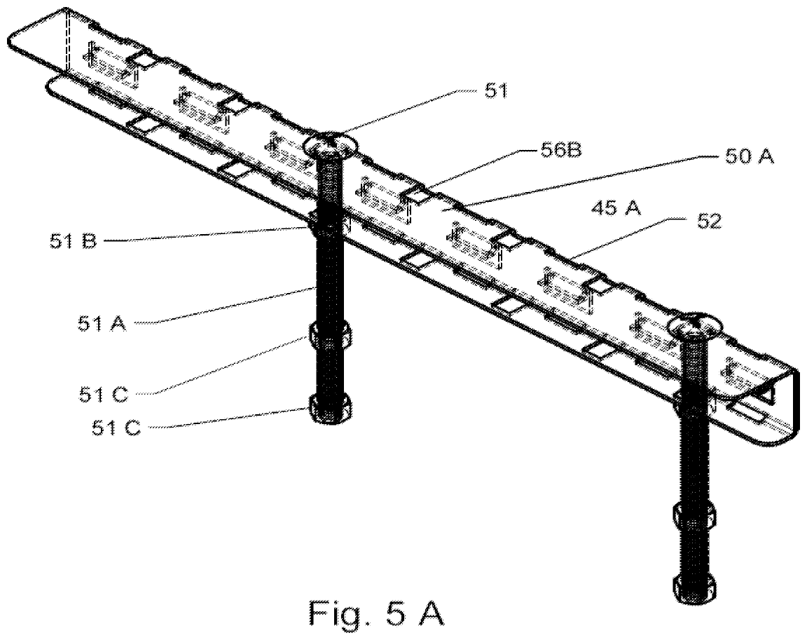

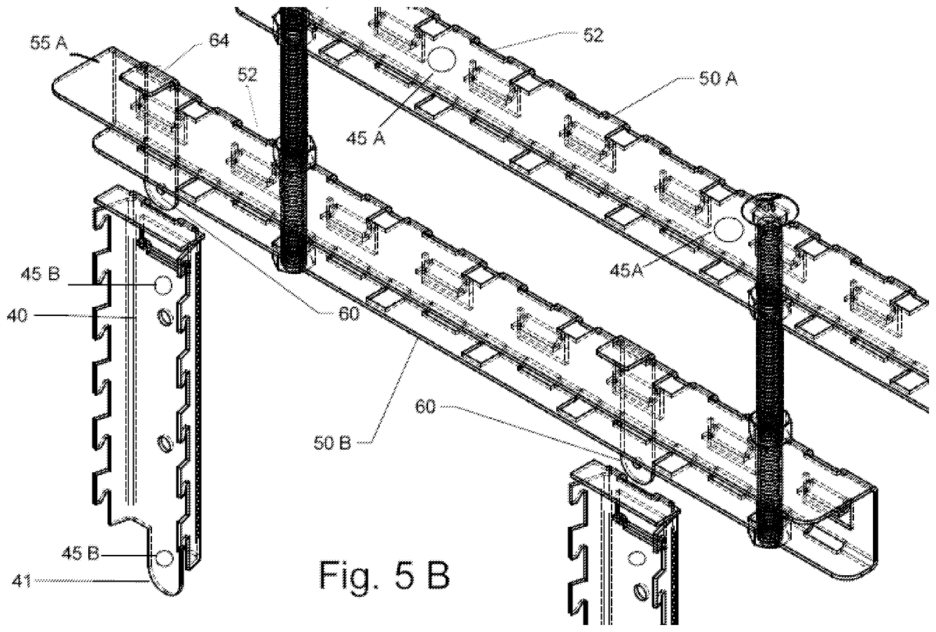

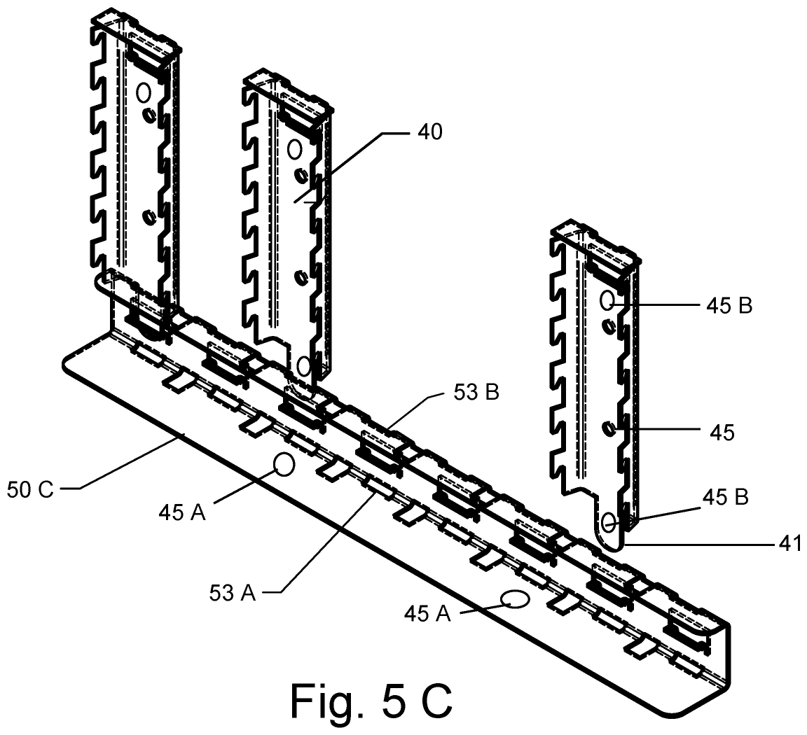

FIGS. 5 through 5C are perspective views of the universal design crossbar 50 shown in FIG. 1 and FIG. 3.

FIG. 5 shows said universal design crossbar 50 comprising an interlocking surface 55A, a wall brace surface 55B, a ceiling/floor brace surface 55C, a plurality bolt holes 56A and 56B, a plurality of prong slots 57A and 57B and a plurality of flanged prong slots 57C. The floor/ceiling mounting holes 45A can be used in a plurality of embodiments.

FIG. 5A shows said crossbar 50 positioned as a stabilizer 50A comprising a flat spacer bolt head 51 on a fully threaded spacer bolt 51A, secured by means of a stabilizer hex nut 51B and two header hex nuts 51C. The parallel vertical threaded spacer bolts 51A are inserted in spacer assembly holes 56B in the stabilizer 50A. Said spacer bolts 51A have means to adjust the distance or space between the stabilizer 50A and header 50B (shown in FIG. 5B) by variably rotating each of the threaded hex nuts 51B on each spacer bolt 51A (shown in FIG. 5B). The top security slot 52 is further shown in FIG. 5B as means to interlock with post segments 40. The floor/ceiling mounting holes 45A and the prong interlocking holes 45B can be used in a plurality of embodiments.

FIG. 5B shows said crossbar 50 positioned as a header 50B with means to interlock the stabilizer 50A by a plurality of spacer bolts 51A and hex nuts 51C to a plurality of post segments 40 each by means of a discrete prong 60. The header 50B interlocks with the adjacent lower post segment 40 by means of a single discrete prong 60 inserted in the top security slot 52 of the header 50B which extends into the lower post segment 40 by means of the top security slot 42 and further extends to the center security slot 43. The floor/ceiling mounting holes 45A can be used in a plurality of embodiments.

The discrete prong 60 has means to interlock a header flange 64 by abutting the interlocking surface SSA of the header 50A. The discrete prong 60 and associated post segments 40 can be positioned in alternative top security slots 52 in the header 50B.

FIG 5C shows said crossbar SO positioned as a footer SOC with means to interlock a plurality of post segments 40 by means of the integrated prong 41 inserted into the footer bottom prong slot 53B with optional use of top prong slot 53A for repositioning. The post segment prong interlocking hole 45B and the footer floor/ceiling mounting holes 45A can be used in a plurality of embodiments.

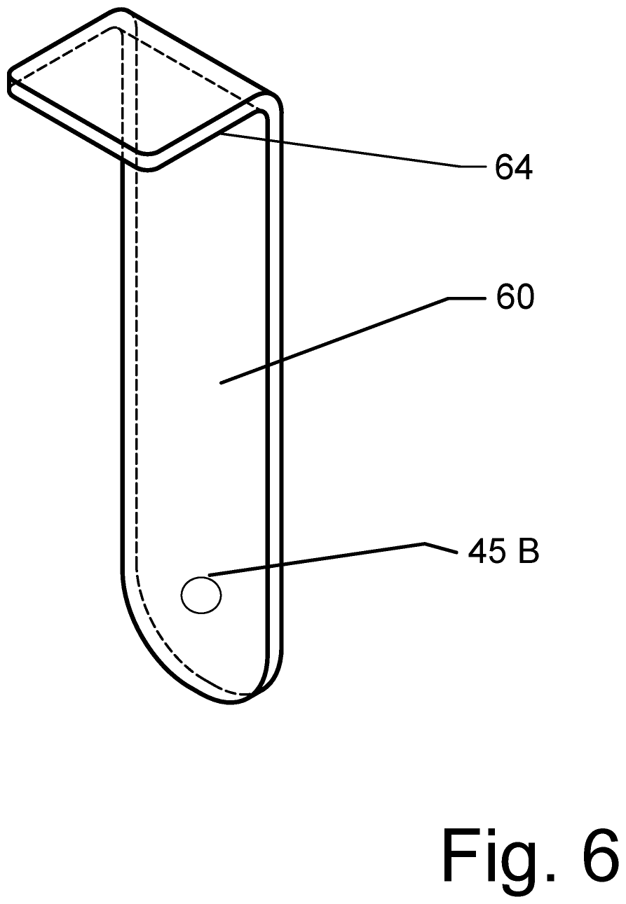

FIG. 6 is a perspective view of the discrete prong 60 shown in FIG. 1, FIG. 3 and FIG. 5B having means to interlock the header 50A with the post segment 40 positioned below the header 50A. The discrete prong 60 enters the top of the post segment 40 through the top security slot 42 and extends into the center security slot 44. The discrete prong header flange 64 abuts the interlocking surface SSA (shown in FIG. 5B) of the header 50A. The prong bolt hole 45B can be used in a plurality of embodiments.

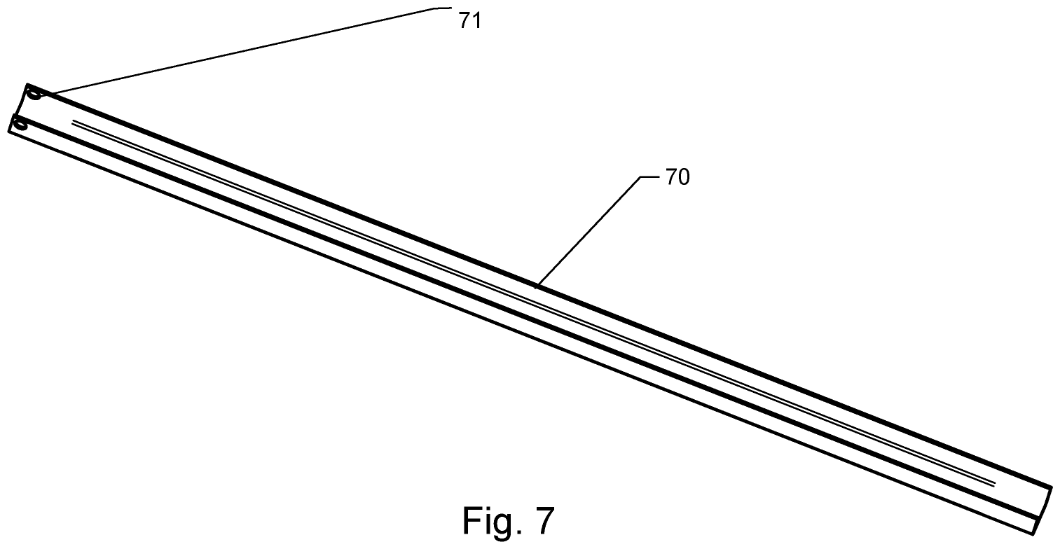

FIG. 7 is a perspective view of the horizontal elongated shelf rail 70 as shown in FIG. 1 and FIG. 3 having means to interlock the parallel spaced rail gripping grooves 46 of a plurality of post segments 40. The shelf rail 70 provides means for the adjustable shelf 20 (shown in FIG. 2) to slide in and out of the cavity. The shelf rail 70 can slide together with the shelf 20. The shelf rail 70 has the extension security lock hole 71 located near the rear of the shelf rail 70.

Operation

In operation, one uses the storage organizer in the first embodiment in upright freezers and refrigerators in place of fixed shelves that are manufacturer factory-installed for home and commercial food service. User removes the factory-installed fixed shelves from the freezer and/or refrigerator, leaving a partially or fully empty cavity. Thus, since the organizer replaces the factory-installed fixed shelves with the user's desired number and position of adjustable, extendable shelves and rails, it avoids the capacity and location restrictions of some prior-art storage organizers.

To replace the factory-installed fixed shelves, user creates two free-standing side panels 30 for which user does not require any tools in the first embodiment. User can create two or more interlocked assembled posts 49 for each side panel 30 for desired load-bearing capabilities with each vertical post 49 comprising a plurality of interlocking post segments 40.

User first interlocks a plurality of vertical post segments 40 into two or more separate assembled posts 49 for each side panel 30 and then interlocks the post segments 40 in each post 49 with horizontal universal design crossbars 50 used variably as: (a) stabilizer 50A, (b) header 50B and (c) footer 50C, thus creating two free-standing side panels 30 for each of the left and right side of the cavity.

User then interlocks a plurality of elongated horizontal shelf rails 70 on parallel vertical post segments 40 by means of rail gripping grooves 46 to populate as user desires, the ladder-like side panel 30 for each of the left-side and right-side of the cavity.

User positions the stabilizer 50A on each side panel 30 to provide a resistance fit with the ceiling of the cavity without damaging the ceiling by means of pressing the ceiling/floor brace surface 55C upward and aligning the stabilizer 50A with optional plurality of corresponding lower headers 50B by means of two or more parallel vertical threaded spacer bolts 51A inserted in spaced bolt holes 56A in the stabilizer 50A and then into corresponding bolt holes 56B in the header 50B, with said bolts 51A providing means to adjust the distance between the stabilizer 50A and header 50B by means of hand-positioning each rotating hex nut 51B and 51C at the bottom of each bolt 51A. The header 50B interlocks with each lower post segment 40 by means of a discrete prong 60 inserted in the top security slot 52 of the header 50B.

User then positions the lower portion of each assembled side panel 30 on the corresponding footer 50C to meet the inside bottom of each left-side and right-side wall in the cavity to provide a resistance fit with the floor of the cavity without damaging the floor, by means of pressing downward the ceiling/floor brace surface 55C.

User adjusts the width of each shelf 20 by means of the left-side extender 22 and right-side extender 23 and adjusts the depth of the shelf 20 by means of the back extender 24. The shelf 20 is easily cleaned, resists physical impact and corrosion, handles heavy use and withstands most environmental conditions.

The back extender 24 is thereby positioned to fit the depth of the cavity and to prevent stored items from sliding off the rear of the shelf 20 by means of a back extender security flange 24B that is angled upwards, having a rigid security tab 24C that can be positioned into various spaced shelf base plate positioning slots 21B in the shelf above to help stabilize the shelf 20 when shelves are closely spaced and are pulled partially out of the cavity thereby helping to prevent the shelf from sliding out uncontrolled.

User populates the two free-standing side panels 30 by means of sliding a plurality of shelves 20 with each positioned in the cavity onto any two parallel rails 70. User can pull out the shelf 20 partially or fully to put items in desired positions and then slide shelf fully inside cavity. The shelf rail 70 has the extension security lock hole 71 located near the rear of the shelf rail 70 as means to prevent the shelf rail 70 from completely sliding out of the cavity along with its associated shelf 20 by means of a plurality of optional locking devices (not shown) inserted in the lock hole 71.

User can stabilize shelf 20 in a partially pulled-out position by means of aligning the back extender security tab 24C in one of the adjacent spaced base plate positioning slots 21B. Rail 70 has an extension security lock hole 71 located near the rear of the rail 70 as means to prevent the rail from completely sliding out of the cavity along with its associated shelf 20 by inserting a plurality of optional locking devices (not shown) in the lock hole 71.

User can accommodate location of factory-installed inner protrusions in the cavity such as dials, knobs, ducts and other devices by means of positioning shelves 20, posts 49 and rails in side panels 30. User can add or decrease storage capacity by inserting or removing a plurality of rails 70 and shelves 20. User positions associated shelf label 25B in the front label holder 25 and in packaging of items to designate position and description of stored items.

When user creates its desired structure of the organizer by assembling required number of post segments 40, rails 70 and shelves 20 with label holder 25 and shelf labels 25B and inserts organizer in the cavity of the freezer and/or refrigerator or other enclosed compartment, six effects increase storage efficiency and capacity:
1) User can pull out shelf partially on rails without user holding shelf in position and without items falling off shelf, and
2) User can slide shelf out fully to relocate shelf on other parallel rails or put shelf elsewhere outside the cavity of the compartment, and
3) User can adjust location of each shelf and separation from another shelf according to item dimensions, weight, and user preference, and
4) User can adjust location of shelf labels designating category and description of items for identifying and retrieving items, and
5) User can position side panels, posts, rails, shelves and shelf extensions to accommodate location of protruding manufacturer-installed controls (e.g., temperature adjustment dials, ducts) within the cavity, and
6) User can monitor quantity and description of stored items by means of optional encoded labels for remote inventory control tracking systems provided for the organizer.

User can view, identify, rearrange and remove stored items on the organizer with ease and flexibility while having air flowing freely around items within the cavity of freezer and/or refrigerator in the first embodiment whereby users can lead a healthier, more relaxed lifestyle and save food costs by using their organizer and labels to locate misplaced items, reduce food spoilage, buy food in bulk at reduced prices and batch cook with healthy ingredients, portion- packed to accommodate varying meal choices on demand by family members (including babies and pets).

Conclusion, Ramifications and Scope

There are 10 million standalone upright freezers currently in homes in the USA of which approximately 35% are the most popular 20 Cubic Foot size. Additionally, most households have a freezer attached to their refrigerator as a side-by-side or as a top or bottom unit.

Over 8 million refrigerators are sold in the U.S. each year and occupy the major share in the home appliances market in sales volume. About 27 percent of today's urban homes and 40 percent of rural ones have at least two refrigerators. Those numbers will likely change again as the COVID-19 pandemic continues to affect lifestyles regarding food storage and consumption in and out of homes.

Worldwide, there are 52.6 million standalone freezers plus 150 million refrigerators. The U.S. market represents 41.9% of the world marketplace.

The COVID-19 pandemic led to a sudden surge in home refrigerator and freezer purchases as people were increasingly stocking supplies, hoarding food and other consumable items which created a huge demand for standalone freezers and refrigerators, thus causing temporary retailer out-of-stock situations, further exacerbated by long shipping and handling times for appliances and manufacturing supplies.

Collectively, there is universal great frustration with the difficulty in finding items easily and quickly in freezers and refrigerators and in throwing away lost expired food. Busy lifestyles lead to over-use of processed unhealthy food with increased interest in healthier food choices, preparation and storage.

The organizer provides a cost-effective and efficient method to solve the above issues, including improved lifestyles and healthier food choices by encouraging low-cost bulk buying and batch cooking using nutritious ingredients resulting in varied meal choices on demand by all family members.

Manufacturing the organizer for a frozen environment requires different methods and more sophisticated and expensive materials than are used in conventional room-temperature storage. This is primarily why this product has not previously been available. Still further advantages will become apparent from a study of the description and the accompanying drawings.

Although the description above contains many specificities, these should not be construed as limiting the scope of the embodiments but as merely providing illustrations of one of the embodiments. For example, in addition to cold and frozen environments, the organizer can be used a plurality of sizes of room-temperature and heated environments; the organizer side panels can be fastened to a wall using one or more of the wall mounting holes (45), to the ceiling and/or floor with the floor/ceiling mounting holes (45A) and post segments can be further interlocked with the prong interlocking holes (45B) in one or more of the post segments (40) and discrete prongs (60).

Thus, the scope of the embodiments should be determined by the appended claims and their legal equivalents, rather than by the examples given.

1. An article of storage organization, said article comprising:
a. A plurality of rigid vertical supports each having a pre-determined cross-sectional shape including a plurality of integrated support methods providing means for interlocking adjacent said vertical supports and horizontal supports, and
b. A plurality of rigid horizontal supports of pre-determined cross-sectional shape providing means for interlocking a plurality of different vertical supports in the article, and
c. A plurality of rigid fasteners providing means for interlocking said horizontal and vertical supports to accommodate multiple configurations, and
d. A plurality of sheets of rigid material of sufficient size and shape to accommodate use by a human being for supporting items of varying dimensions and weights and having means to easily adjust the position of said material on the horizontal and vertical supports, and
e. A plurality of rigid elongated horizontal supports of varying dimensions each providing means for interlocking said sheets with a plurality of vertical supports, and
f. A plurality of flexible and rigid material of sufficient size to identify said items and to be easily positioned on the sheets of rigid material and in packaging of the items,
Whereby a human being can conveniently and easily store, organize, rearrange, view, identify and retrieve a variety of said items.

2. The article of storage organization in claim 1 wherein said rigid vertical and horizontal supports can be made of, but are not limited to, corrosion-resistant steel and aluminum material.

3. The article of storage organization in claim 1 wherein said sheets of rigid material for supporting items can be made of, but are not limited to, thermoplastic polymer material, including synthetic resin, polypropylene and polyester.

4. The article of storage organization in claim 1 wherein each said sheet can include one or more pre-determined moveable sections each with means to interlock with said sheet at spaced locations so as to be adjusted to fit varying depths and widths wherein the article of storage organization is located.

5. The article of storage organization in claim 1 wherein said flexible or rigid material to identify items can be made of, but is not limited to, thermoplastic polymer material.

6. The article of storage organization in claim 1 wherein said flexible or rigid material to identify items can be encoded for visual and electronic identification of quantities, content and locations of said items.
